(19) United States
(12) Patent Application Publication
(10) Pub. No.: US 2014/0330241 A1
(43) Pub. Date: Nov. 6, 2014

(54) INFUSION SYSTEM WITH RAPID ACCESS TO CODE MEDICATION INFORMATION

(71) Applicant: CAREFUSION 303, INC., San Diego, CA (US)

(72) Inventors: Stephen Bollish, San Diego, CA (US); Jeffrey L. Gaetano, San Diego, CA (US)

(73) Assignee: CareFusion 303, Inc., San Diego, CA (US)

(21) Appl. No.: 13/887,260

(22) Filed: May 3, 2013

(51) Int. Cl. A61M 5/142; G06F 3/0484; A61B 19/00

(52) U.S. Cl. CPC A61M 5/142; A61B 19/56; G06F 3/04842
USPC 604/500; 604/151; 715/771

(57) ABSTRACT

The subject matter described herein relates to a provision of rapid access to one or more medications relevant for treating a patient-related code event of a patient. A user interface device can concurrently display medications available in a hospital and a code medications button. When the user interface device receives a clinician-generated input characterizing a selection/click of the code medications button, the user interface device can display a set of medications generally used to treat the patient-related code event, such as cardiac arrest. Related apparatus, methods, systems, techniques and articles are also described.

### TECHNICAL FIELD

The subject matter described herein relates to an infusion system (for example, an infusion pump) that provides, on a graphical user interface, rapid access to information for one or more medications associated with a code event.

### BACKGROUND

Infusion systems, such as infusion pumps, are typically customized for each particular care area within a hospital or other healthcare facility. For example, infusion pumps residing within a neonatal intensive care unit (NICU) can be configured to administer a different set of medications and at different dosing levels/rates as compared to a general surgery care area that handles adult patients. Furthermore, each care area within the hospital can have different protocols to address code events. The Advanced Cardiovascular Life Support (ACLS) protocol for pediatric care areas differs from ACLS used for adult patients in other care areas within the hospital.

### SUMMARY

The current subject matter describes an infusion system that provides rapid access to one or more medications relevant for treating a patient-related code event, which can be a life-threatening event such as cardiac arrest. A user interface device can concurrently display medications available in a hospital care area and a code medications button (or other type of graphical user interface element). When the user interface device medications button, the user interface device can display a set of medications generally used to treat the life threatening patient-related code event.

In one aspect, a plurality of medications from a first medication set and a graphical user interface element can be concurrently displayed in a user interface device of an infusion system. The graphical user interface element can be associated with a plurality of medications from a second medication set. The second medication set can be used to treat a clinical intervention for at least one patient-related code event. A clinician-generated input selecting the graphical user interface element can be received at the user interface device. In response to the clinician-generated input, the user interface device can display information characterizing one or more of the medications from the second medication set.

In some variations, one or more of the following can be optionally implemented. The first medication set can include medications specific for a healthcare area within a hospital. The graphical user interface element can be a code medication button. The second medication set can include code medications used to treat patients diagnosed with the at least one patient-related code event. The at least one patient-related code event can include one of: a cardiac arrest, a respiratory arrest, a seizure, a stroke, and any other life-threatening event. In one implementation, the at least one patient-related code event can be cardiac arrest, and the second medication set can include Advanced Cardiovascular Life Support (ACLS) drugs including one or more of: epinephrine, vasopressin, amiodarone, procainamide, dopamine, sotalol, lidocaine, magnesium sulfate, adenosine, atropine, nitroglycerin, morphine, sodium bicarbonate, calcium blockers, angiotensin-converting-enzyme (ACE) inhibitors, glycoprotein inhibitors, anticonvulsants and fibrinolytic therapy medications.

The displayed medications of the second medication set can be retrieved for display from a database connected to the user interface device via a communication network. The database can store a mapping between the at least one patient-related code event and the second medication set.

Further, a selection by a clinician of at least one medication from the second medication set and a specification of delivery data for delivery of the at least one selected medication can be received at the user interface device. The delivery data can include a quantity of the at least one selected medication that is to be delivered, and a flow rate of the delivery of the at least one selected medication. A controller connected to the user interface device can actuate a delivery of the at least one selected medication to a patient in accordance with the delivery data. At least one delivery device connected to the controller can deliver the one or medications to the patient. The at least one delivery device can obtain the selected medication from a medication storage device connected to the controller to deliver the medication to the patient. The user interface device can send an identification of the selected one or more medications to a medication storage and delivery system that sends the selected at least one medication to the medication storage device. The user interface device can be connected to the medication storage and delivery system via a communication network.

In another aspect, a system is described that can include a user interface device, a controller, and a delivery device. The user interface device can: receive a preference for display of medications based on one of a plurality of medications, favorite medications from the plurality of medications, and code medications from the plurality of medications. The user interface device can display medications associated with the preference. The user interface device can receive a selection of a medication from the displayed medications and a delivery data. The controller can receive the delivery data from the user interface device. The delivery device can be actuated by the controller to deliver the selected medication in accordance with the delivery data.

In some variations, one or more of the following can be optionally implemented. The plurality of medications can characterize all medications available in a healthcare area within a hospital where the patient is being treated. The favorite medications can characterize medications that a clinician may prefer and may often provide to associated patients. The code medications can characterize medications that are usually delivered to patients diagnosed with a health condition treated in a corresponding healthcare area where the clinician works and where the patient is being treated. The healthcare area can be one of a plurality of healthcare areas within the hospital.

The system can further include a database that can be connected to the user interface device via a communication network. The database can store at least one of a mapping between each medication of the plurality of medications and a corresponding healthcare area of a plurality of healthcare areas where the patient is being treated, the favorite medications of a clinician using the user interface device, and the code medications for the corresponding healthcare area. The user interface can retrieve medications associated with the preference of display from the database. The retrieved medications can be displayed on a graphical user interface of the user interface device. The delivery data can include at least one of: a quantity of the selected medication that is to be delivered and a flow rate of the delivery of the selected medication.

The system can further include a medication storage device to store the selected medication. The delivery device can obtain the selected medication from the medication storage device to deliver the medication to the patient. The system can further include computational medication cabinets to receive the selection of the medication from the user interface device and deliver the selected medication to the medication storage device. The computational medication cabinets can include a medication ordering system of a pharmacy.

In yet another aspect, an infusion pump is described. The infusion pump can include a user interface device and a controller. The user interface device can receive a preference for display of medications based on one of a plurality of medications, favorite medications from the plurality of medications, and code medications from the plurality of medications. The user interface device can display medications associated with the preference. The user interface device can receive a selection of a medication from the displayed medications and a delivery data. The controller can receive the delivery data from the user interface device. The controller can actuate a delivery device to deliver the selected medication in accordance with the delivery data.

In some variations, one or more of the following can be optionally implemented. The user interface device can be connected to a database via a communication network. The database can store at least one of: a mapping between each medication of the plurality of medications and a corresponding healthcare area of a plurality of healthcare areas where the patient is being treated, the favorite medications of a clinician using the user interface device, and the code medications for the corresponding healthcare area. The user interface can retrieve medications associated with the preference of display from the database. The retrieved medications can be displayed on a graphical user interface of the user interface device. The delivery data can include a quantity of the selected medication that is to be delivered and a flow rate of the delivery of the selected medication.

The infusion pump can further include a medication storage device to store the selected medication. The delivery device can obtain the selected medication from the medication storage device to deliver the medication to the patient. The medication storage device can receive the selected medication from a medication storage and delivery system. The medication storage and delivery system can include computational medication cabinets that form a medication ordering system of a pharmacy.

Computer program products are also described that comprise non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The subject matter described herein provides many advantages. For example, while treating a patient, the user interface device of an infusion system can rapidly display only relevant medications that the clinician would have otherwise searched from an extensive database of medications. Thus, time and effort of the clinician are saved.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating an infusion system that can rapidly display one or more medications relevant for treating a patient-related code event of a patient;

FIG. 2 is a diagram illustrating a graphical user interface displaying available drugs;

FIG. 3 is a diagram illustrating a graphical user interface displaying a status of delivery by at least one delivery device;

FIG. 4 is a diagram illustrating a graphical user interface displaying available fluids;

FIG. 5 is a diagram illustrating a graphical user interface displaying available drugs for a healthcare area where patients with a specific health condition are treated with drugs, and not fluids;

FIG. 6 is a diagram illustrating a graphical user interface displaying favorite drugs when the clinician selects the favorites button;

FIG. 7 is a diagram illustrating a graphical user interface with the favorites button not included when there are no favorites;

FIG. 8 is a diagram illustrating a graphical user interface displaying code drugs when the clinician selects the code drugs button;

FIG. 9 is a diagram illustrating a graphical user interface displaying all available drugs when the clinician selects the all drugs button;

FIG. 10 is a diagram illustrating a graphical user interface that displays the code drugs in the drugs list area when the clinician selects the code drugs button;

FIG. 11 is a diagram illustrating a graphical user interface displaying all available drugs when the clinician selects the all drugs button;

FIG. 12 is a diagram illustrating a graphical user interface displaying all available fluids;

FIG. 13 is a diagram illustrating a transition from a first graphical user interface to a second graphical user interface when the clinician specifies delivery data including the rate of delivery and quantity of delivery;

FIG. 14 is a diagram illustrating a graphical user interface allowing a clinician to select a secondary drug for delivery from a list of all drugs;

FIG. 15 is a diagram illustrating a graphical user interface displaying favorite secondary drugs when the clinician selects the favorite secondary drugs button;

FIG. 16 is a diagram illustrating a graphical user interface displaying all secondary drugs available in the hospital when the clinician selects the all secondary drugs button;

FIG. 17 is a diagram illustrating a graphical user interface displaying code secondary drugs in the secondary drugs list area when the clinician selects the code secondary drugs button;

FIG. 18 is a flow-diagram illustrating a rapid display and subsequent delivery of medications required for treating the patient; and

FIG. 19 is a system diagram illustrating a possible computing landscape for rapidly displaying and subsequently delivering medications required for treating the patient.

Like reference symbols in the various drawings indicate like elements.

### DETAILED DESCRIPTION

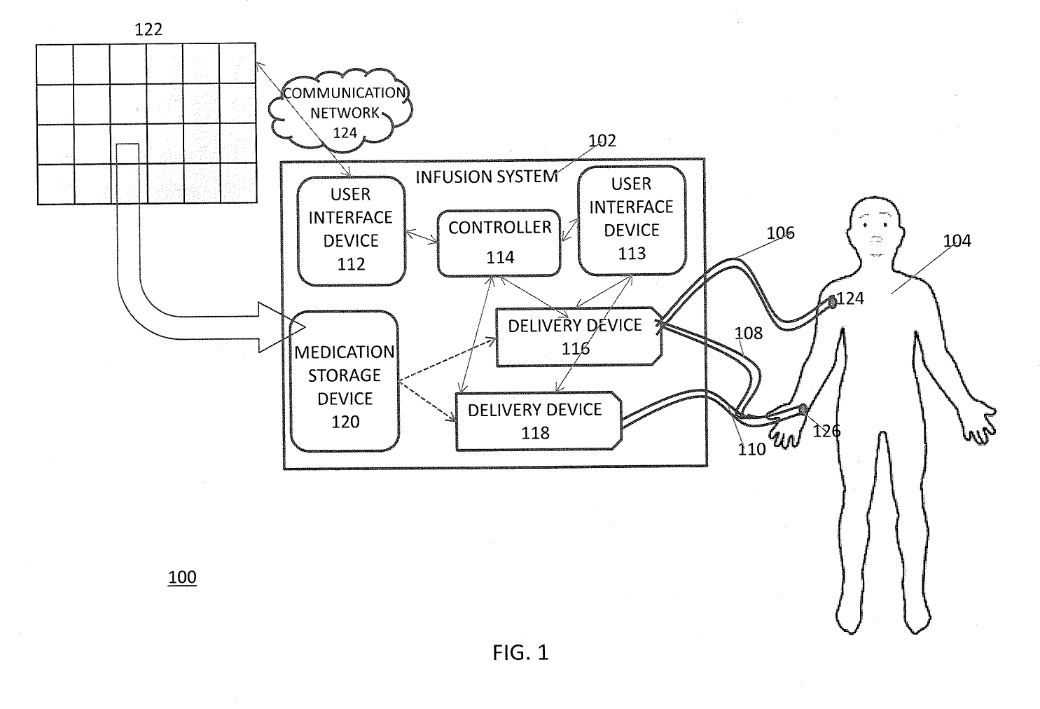

FIG. 1 is a diagram 100 illustrating an infusion system 102 (for example, an infusion pump) that can rapidly display one or more medications relevant for treating a patient-related code event of a patient 104. The infusion system 102 can deliver the selected medication to the patient 104 via infusion channels 106, 108, and 110. The infusion system 102 can include a user interface device 112, a user interface device 113, a controller 114, delivery devices 116 and 118, and a medication storage area 120.

The user interface device 112 can execute graphical user interfaces 202, 402, 502, 6602, 702, 802, 902, 1002, 1102, 1202, 1402, 1502, 1602, and 1702, which are described below. These graphical user interfaces 202, 402, 502, 6602, 702, 802, 902, 1002, 1102, 1202, 1402, 1502, 1602, and 1702 can allow a clinician to search for desirable medications from a medication library that can include a plurality of medications (for example, hundreds or thousands of medications). Based on an input by the clinician, the graphical user interface device 112 can display at least one of the following: all medications available in a hospital; medications searched by a clinician in accordance with a search criteria (for example, generic name of medication, and the like) specified by a clinician; favorite medications characterizing medications that the clinician may prefer and/or may often provide to patients; code medications characterizing medications that are usually delivered to patients diagnosed with an eminently life threatening health condition treated in a healthcare area (within a hospital); a dose calculator to calculate dosage of the medication to be delivered to the patient 104; and a rate of delivery of the medication that is to be delivered to the patient 104. The medications can be drugs and/or fluids, wherein the drugs can be in the form of a solution or other forms.

The user interface device 112 can receive, from a clinician, a selection of a medication from the displayed medications. When the user interface device 112 receives the selection of the medication, the user interface device 112 can send an identification (for example, name and/or a unique identifier) of the selected medication to a medication storage and delivery system 122 (for example, computational medication cabinets) via a communication network 124. In response, the medication storage and delivery system 122 can send the selected medication to the medication storage device 120, which can store the selected medication.

The user interface device 112 can send the delivery data (for example, rate of delivery of the medication) specified by the clinician to the controller 114. Based on the delivery data, the controller 114 can actuate one of the delivery device 116 and the delivery device 118 to deliver the medication stored in the medication storage device 120 to locations 124 and/or 126 on a body of the patient 104 via the infusion channels 106, 108, and/or 110. Some portion of the infusion channel 108 can overlap with some portion of the infusion channel 110. In some alternate implementations, the clinician can manually deliver the medication from the medication storage device 120 to the patient 104. This manual delivery can be either an oral delivery or an injection/infusion.

The clinician can be a doctor or a nurse. In some variations, the clinician can be a pharmacist, an assistant or associate in a hospital or laboratory, a psychologist, a psychiatrist, and/or any other authorized individual. In some implementations, the clinician can be the patient 104.

The user interface device 112 can be a computer or a tablet computer. The user interface device 112 can be configured to receive input from the clinician and can be configured to display an output to the clinician. To receive input, the user interface device 112 can be a touch screen device or can be attached with an input device, such as a mouse, a joystick, a keyboard, a voice detection device, or any other input device. To provide output, the user device 112 can include a display screen, which can be a cathode ray tube (CRT) monitor, a liquid crystal display (LCD) monitor, a light emitting diode (LED) monitor, or any other display screen.

The controller 114 can be one or more of: one or more microcontrollers, one or more microprocessors, one or more computers, one or more servers, and the like.

The delivery devices 116 and 118 can be mechanical devices that can deliver medications to a body of the patient 104 via the infusion channels 106, 108, and 110. The controller 114 can control the activations and movements of the delivery devices 116 and 118. In some implementations, the delivery devices 116 and/or 118 can include a plunger and syringe. The controller 114 can turn a screw that can push on the plunger in accordance with the infusion channel identifiers and time durations specified by the clinician. In some implementations, the controller 114 can be embedded with at least one of the delivery devices 116 and 118.

The infusion channels 106, 108, and 110 can have circular or oval cross-sections. Although circular and oval cross-sections are described, in other implementations, the infusion channels can have other shapes of cross-sections, such as a square, a rectangle, a triangle, a hexagon, a polygon, or any other shape. The infusion channels can be made of rubber, silicone, or any other material.

The clinician can deactivate one or more infusion channels 106, 108, and 110 by removing at least a portion of the tubing forming the infusion channel. In other implementations, to deactivate an infusion channel 106, 108, or 110, the clinician can occlude the infusion channel by placing a clamp or stopcock in the infusion channel such that the desired infusion channel is occluded while not affecting other infusion channels. The clamp can be one or more of: a roller clamp, a slide clamp, a stopcock and any other such device. To activate or re-activate an infusion channel 106, 108, or 110, the clinician can remove the clamp from the infusion channel.

The medical storage and delivery system 122 can be computational medication cabinets that can store a plurality (for example, tens, hundreds, thousands, or more) of medications. In other implementations, the medical storage and delivery system can be a medication ordering system of a pharmacy, a storage and online medication delivery system, and the like.

Although three infusion channels 106, 108, and 110 have been described, in some implementations, one or more than two infusion channels can be used. Although two delivery devices 116 and 118 have been described, in other implementations, one or more than two delivery devices can be used. Although two locations 124 and 126 on a body of the patient 104 have been described, in alternate implementations, one location or more than two locations can be present.

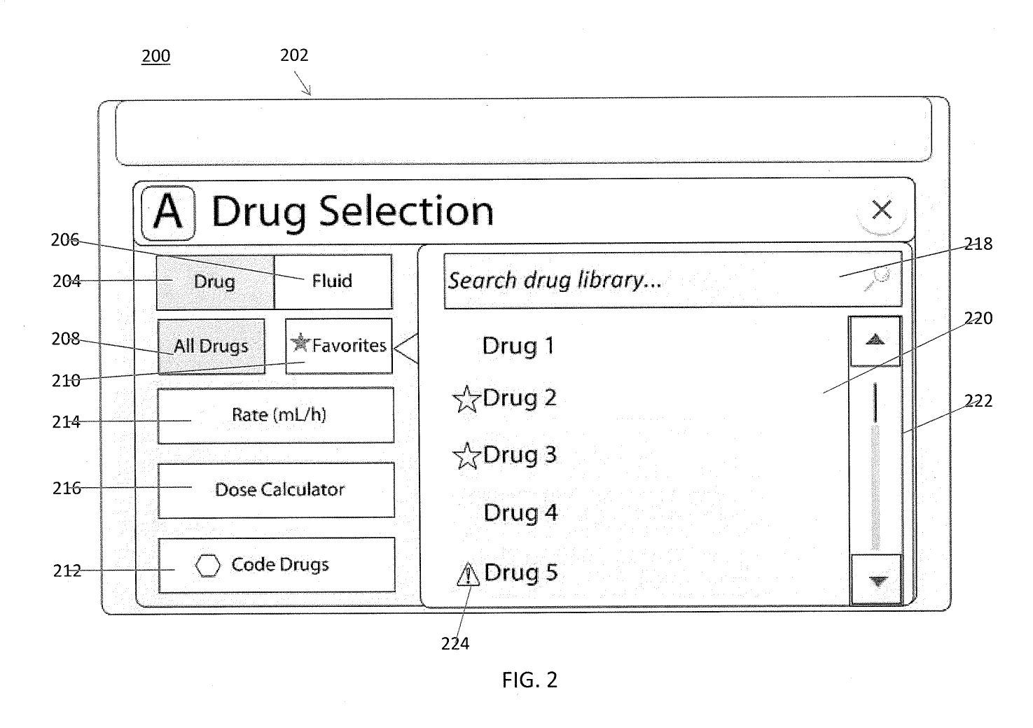

FIG. 2 is a diagram 200 illustrating a graphical user interface 202 displaying available drugs. These available drugs can be all the drugs available in the hospital. The user interface device 112 can execute the graphical user interface 202. The graphical user interface 202 can display a drug button 204, a fluid button 206, an all drugs button 208, a favorites button 210, a code drugs button 212, a rate button 214, a dose calculator button 216, a search area 218, a drugs list area 220, and an optional scrollbar 222.

When the clinician begins using the user interface device 112, the drug button 204 can be selected as default. When the drug button 204 is selected by default or when the clinician selects the drug button 204, the user interface device 112 can display data associated with drugs on the graphical user interface 202. When the clinician selects the fluid button 206, the user interface device 112 can display the data associated with fluid medications on a fluid selection graphical user interface, such as the graphical user interface 402 (described below) or the graphical user interface 1202 (described below).

The user interface device 112 can display, by default, all the drugs available in the hospital in the drugs list area 220. When the clinician clicks the all drugs button 208, the user interface device 112 can display all the drugs in the drugs list area 220.

When the clinician clicks the favorites button 210, the user interface device 112 can display only the favorite drugs in the drugs list area 220. The favorite drugs can characterize drugs that the clinician may prefer and may often provide to patients. The favorite drugs can be displayed along with a common icon (for example, an icon showing a star) indicating that the associated drug is a favorite drug.

When the clinician clicks the code drugs button 212, the user interface device 112 can display only the code drugs in the drugs list area 220. The code drugs can characterize drugs that are usually delivered to patients diagnosed with a health condition treated in the healthcare area (within a hospital) where the clinician works. The code drugs can be displayed along with a common icon (for example, an icon showing a hexagon) indicating that the associated drug is a code drug.

All available drugs can include critical drugs that can be displayed with another common icon, such as an icon 224 showing an exclamation mark. The critical drugs can characterize drugs that can be delivered to the patient 104 when the condition of the patient 104 is critical.

The search area 218 can allow the clinician to search for a drug from the drugs displayed in the drugs list area 220. In another implementation, the search area 218 can allow the clinician to search for a drug from a drug library, which can include all the drugs available in the hospital. The drug library can be a portion of the medication library.

When the drugs are displayed in the drugs list area 220, the user interface device 112 can display drugs in an alphabetical order. In another implementation, the user interface device 112 can display drugs in the following order: critical drugs in an alphabetical order, favorite drugs in an alphabetical order, code drugs in an alphabetical order, and then remaining drugs in alphabetical order. In yet another implementation, the clinician can specify any order for the drugs displayed in the drugs list area 220.

When all the drugs in the drugs list area 220 extend beyond a display area (for example, one page) that can be seen by the clinician, the graphical user interface 202 can include a scrollbar that can be used to view all the displayed drugs. In alternate implementations, the graphical user interface 202 can allow the clinician to swipe a touchscreen to scroll though the list, as performed on some smart phones, such as an IPHONE.

When the clinician clicks the rate button 214, the user interface device 112 can direct the clinician out of medication selection and to another graphical user interface where the user can specify a basic infusion, such as a simple rate/volume infusion without specifying upper and lower limits of the medication. When the clinician clicks the dose calculator button 216, the user interface device 112 can direct the clinician out of medication selection to another graphical user interface where the user can use a basic drug calculation feature.

While the functions above have been described for drugs, similar functions can be performed for fluids when the clinician clicks the fluid button 206.

In some alternate implementations, the clinician can disable one or more of the drug button 204, the fluid button 206, the all drugs button 208, the favorites button 210, the code drugs button 212, the rate button 214, the dose calculator button 216. The user interface device 112 may not display the disabled buttons, as shown by the graphical user interfaces 402, 502, 602, 702, 802, 902, 1002, 1102, 1202, 1402, 1502, 1602, and 1702, which are described below.

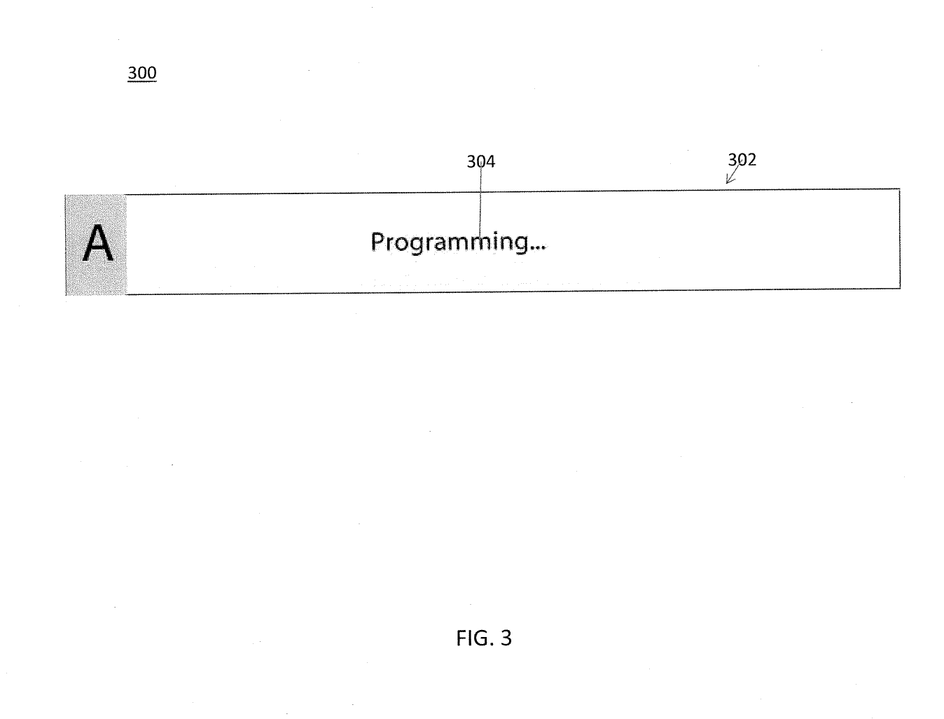

FIG. 3 is a diagram 300 illustrating a graphical user interface 302 displaying a status of delivery by at least one of the delivery device 116 and/or the delivery device 118. In one implementation, the graphical user interface 302 can be a screen executed on a delivery device or infusion module. In another implementation, the user interface device 113 can execute the graphical user interface 302. When the clinician selects a drug from the drugs list area 220, the graphical user interface 302 can display a message 304 (for example, “Programming . . . ”) until the infusion system 102 begins delivering the selected drug. When the infusion system 102 begins delivering the selected drug, the user interface 113 can execute another graphical user interface (for example, graphical user interface 1404 described below) that can display the rate at which the selected drug is being delivered.

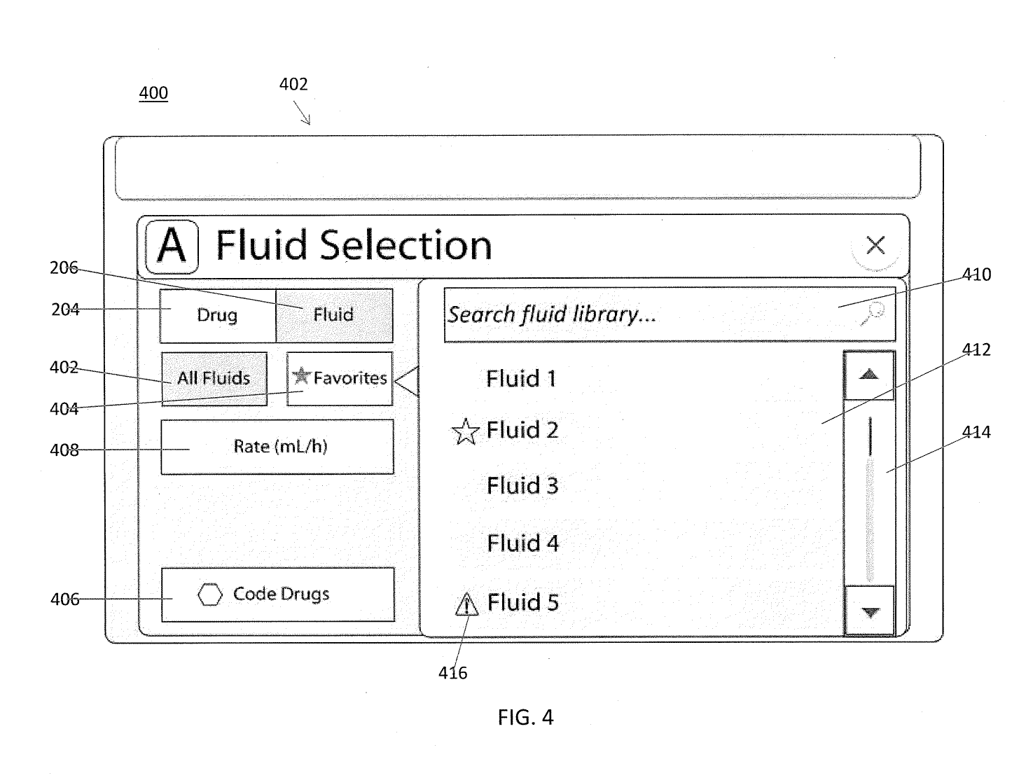

FIG. 4 is a diagram 400 illustrating a graphical user interface 402 displaying available fluids. The user interface device 112 can execute the graphical user interface 402. The graphical user interface 402 can display a drug button 204, a fluid button 206, an all fluids button 402, a favorites button 404, a code fluids button 406, a rate button 408, a search area 410, a fluids list area 412, and an optional scrollbar 414.

When the clinician selects the fluids button 206, the user interface device 112 can display data associated with fluids on the graphical user interface 202. The user interface device 112 can display all the fluids available in the hospital in the fluids list area 412 by default. When the clinician clicks the all fluids button 402, the user interface device 112 can display all the fluids in the fluids list area 412.

When the clinician clicks the favorites button 404, the user interface device 112 can display only the favorite fluids in the fluids list area 412. The favorite fluids can characterize fluids that the clinician may prefer and/or may often provide to patients. The user interface device 112 can display favorite fluids along with a common icon (for example, an icon showing a star) indicating that the associated drug is a favorite drug. While the icons (for example, stars) for favorite drugs have been shown to be same as the icons (for example, stars) for favorite fluids, in some implementations, the icons for favorite drugs can be different from the icons for the favorite fluids.

When the clinician clicks the code fluids button 406, the user interface device 112 can display only the code fluids in the fluids list area 412. The code fluids can characterize fluids that are usually delivered to patients diagnosed with a health condition treated in the healthcare area where the clinician works. The names of code fluids can be displayed along with a common icon (for example, an icon showing a hexagon) indicating that the associated fluid is a code fluid. While the icons (for example, hexagons) for code drugs have been shown to be same as the icons (for example, hexagons) for code fluids, in some implementations, the icons for code drugs can be different from the icons for the code fluids.

All available fluids can include critical fluids that can be displayed with another common icon, such as an icon 416 showing an exclamation mark. The critical fluids can characterize fluids that can be delivered to the patient 104 when the condition of the patient 104 is critical. While the icon 224 for each critical drug has been shown to be same as the icon 416 for each critical fluid, in some implementations, the icon for a critical drug can be different from the icon for the critical fluid.

The search area 410 can allow the clinician to search for a fluid from the fluids displayed in the fluids list area 412. In another implementation, the search area 410 can allow the clinician to search for a fluid from a fluid library, which can include all the fluids available in the hospital. The fluid library can be a portion of the medication library.

When the fluids are displayed in the fluids list area 412, the user interface device 112 can display fluids in an alphabetical order. In another implementation, the user interface device 112 can display fluids in the following order: critical fluids in an alphabetical order, favorite fluids in an alphabetical order, code fluids in an alphabetical order, and then remaining fluids in alphabetical order. In yet another implementation, the clinician can specify any order for the fluids displayed in the fluids list area 220.

When all the fluids in the fluids list area 412 extend beyond a display area (for example, one page) that can be seen by the clinician, the graphical user interface 402 can include a scrollbar that can be used to view all the displayed fluids. In another implementation, the graphical user interface 402 can allow the clinician to swipe a touchscreen to scroll though the list, as performed on some smart phones, such as an IPHONE.

When the clinician clicks the rate button 408, the user interface device 112 can direct the clinician out of medication selection and to another graphical user interface where the user can specify a basic infusion, such as a simple rate/volume infusion without specifying upper and lower limits of the medication. The infusion system 102 can deliver the fluid to the patient 104 at the specified rate.

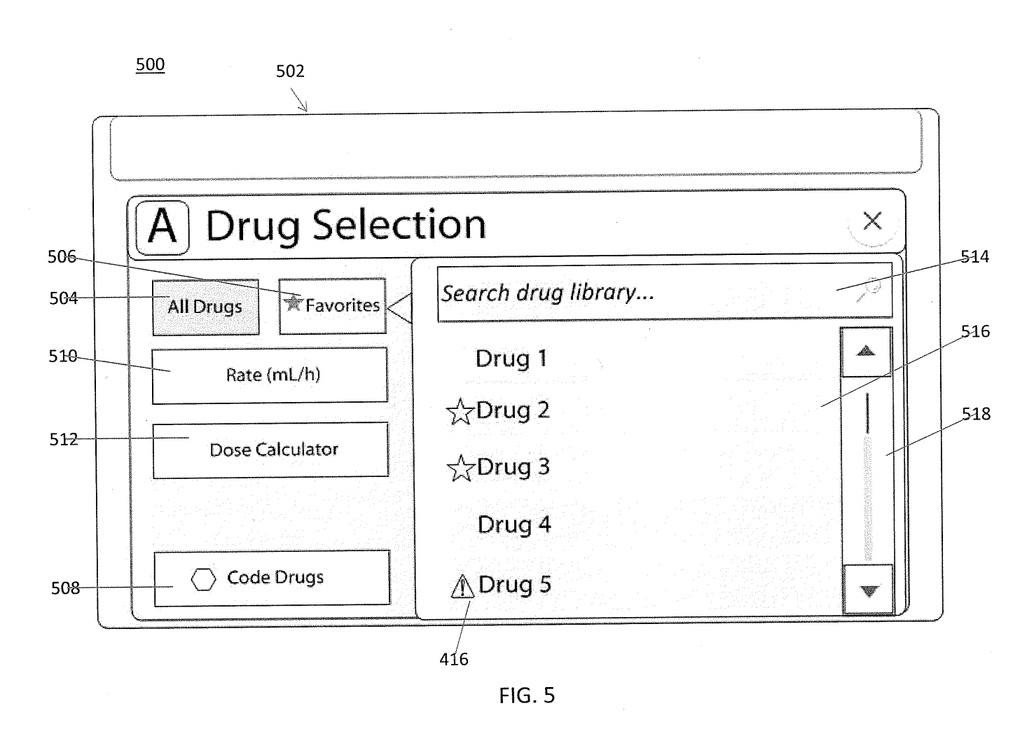

FIG. 5 is a diagram 500 illustrating a graphical user interface 502 displaying available drugs for a healthcare area where patients with a specific health condition are treated with drugs, and not fluids. The user interface device 112 can execute the graphical user interface 502. This healthcare area can be a particular unit/location within a hospital. This healthcare area may not require fluids. Thus, the user interface device 112 can execute a separate graphical user interface for each healthcare area based on requirements of patients treated within the healthcare area.

The graphical user interface 502 can include an all drugs button 504, a favorites button 506, a code drugs button 508, a rate button 510, a dose calculator button 512, a search area 514, a drugs list area 516, and an optional scrollbar 518. When the clinician clicks the all drugs button 504, the user interface device 112 can display all the drugs in the drugs list area 516. When the clinician clicks the favorites button 506, the user interface device 112 can display only the favorite drugs in the drugs list area 516. When the clinician clicks the code drugs button 508, the user interface device 112 can display only the code drugs in the drugs list area 516. All available drugs can include critical drugs.

The search area 514 can allow the clinician to search for a drug from the drugs displayed in the drugs list area 516. In another implementation, the search area 514 can allow the clinician to search for a drug from the drug library, which can include all the drugs available in the hospital. When all the drugs in the drugs list area 516 extend beyond a display area (for example, one page) that can be seen by the clinician, the graphical user interface 502 can include a scrollbar 518 that can be used to view all the displayed drugs.

When the clinician clicks the rate button 510, the user interface device 112 can direct the clinician out of medication selection and to another graphical user interface where the user can specify a basic infusion, such as a simple rate/volume infusion without specifying upper and lower limits of the medication. When the clinician clicks the dose calculator button 512, the user interface device 112 can direct the clinician out of medication selection to another graphical user interface where the user can use a basic drug calculation feature.

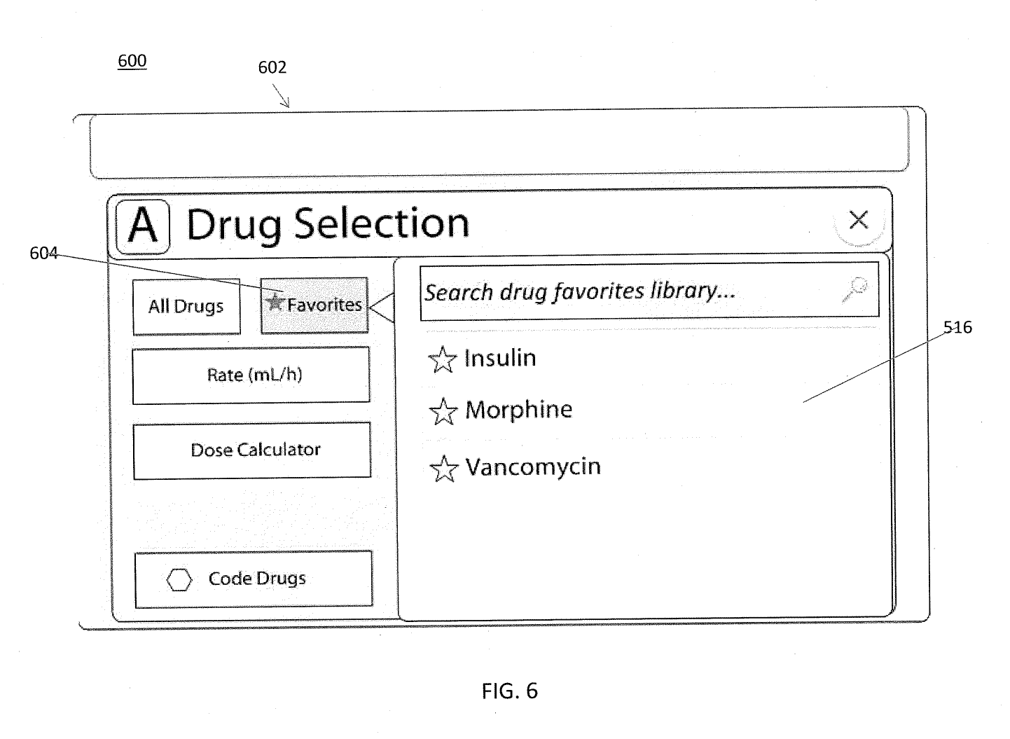

FIG. 6 is a diagram 600 illustrating a graphical user interface 602 displaying favorite drugs when the clinician selects the favorites button 604. The user interface device 112 can execute the graphical user interface 602. When the clinician clicks the favorites button 604, the user interface device 112 can display the favorite drugs in the drugs list area 516. Some examples of the favorite drugs can be insulin, morphine, and vancomycin. Authorized users of the hospital or the healthcare area within the hospital can modify the list of favorite drugs by deleting or adding drugs to this list.

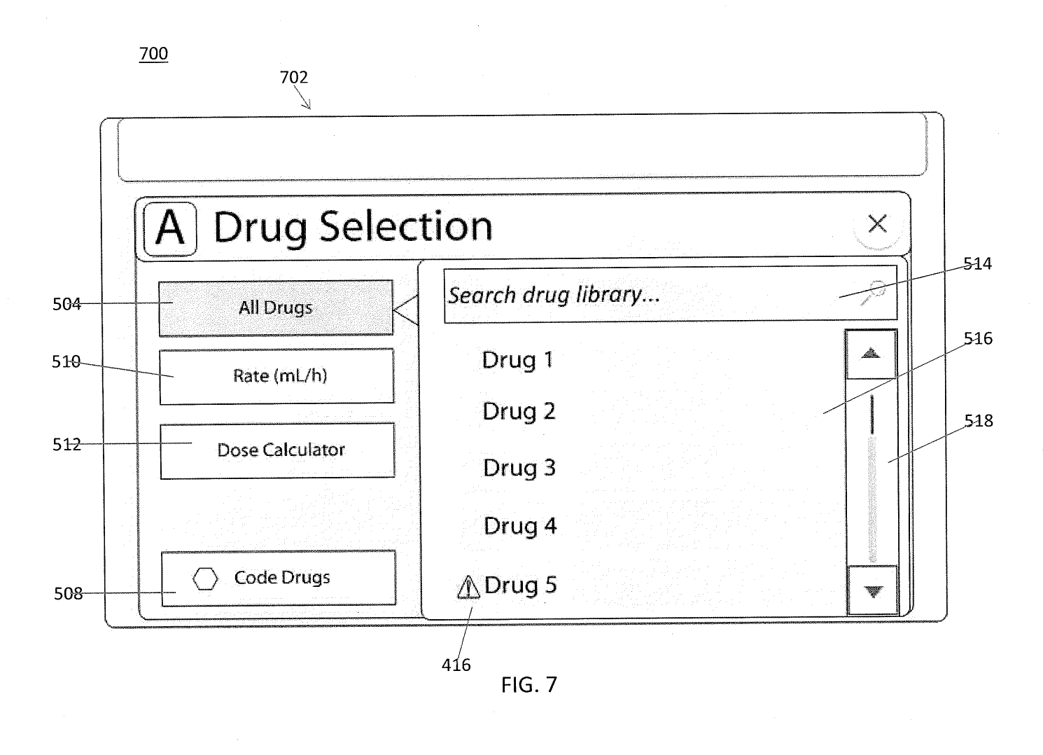

FIG. 7 is a diagram 700 illustrating a graphical user interface 702 with the favorites button not included when there are no favorites. The user interface device 112 may not display one or more disabled buttons.

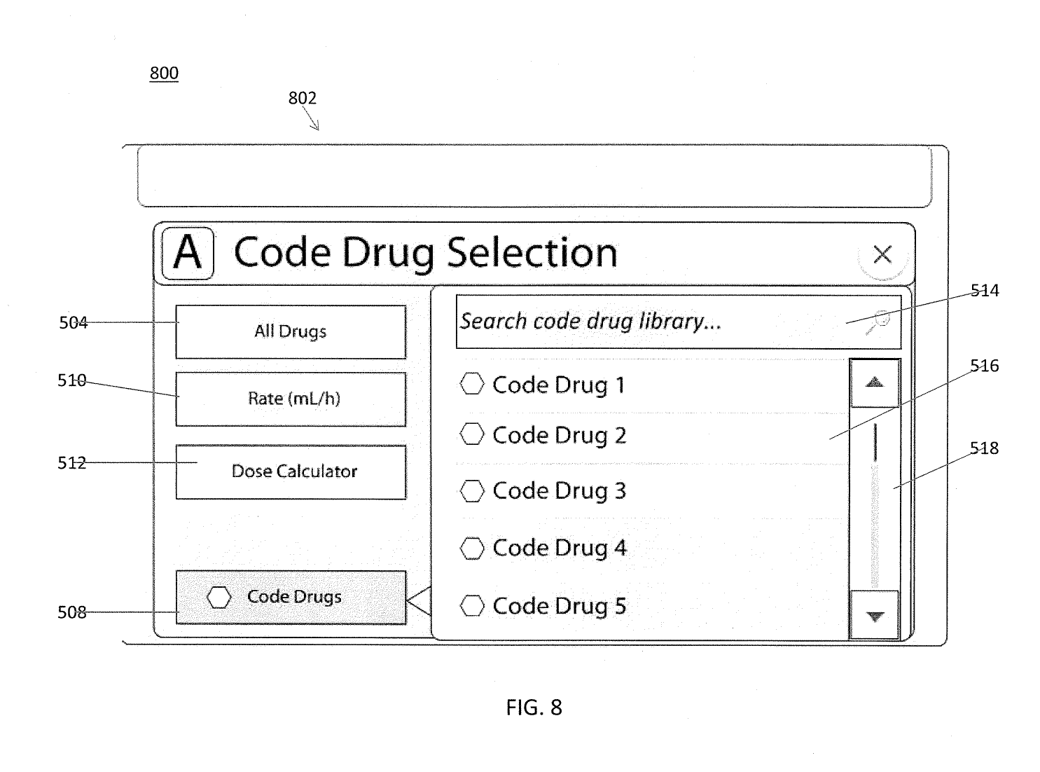

FIG. 8 is a diagram 800 illustrating a graphical user interface 802 displaying code drugs in the drugs list area 516 when the clinician selects the code drugs button 508. The clinician can select a code drug from the drugs list area 516, and can then proceed to specify dose, or rate of delivery of the selected code drug so as to deliver the drug to the patient 104.

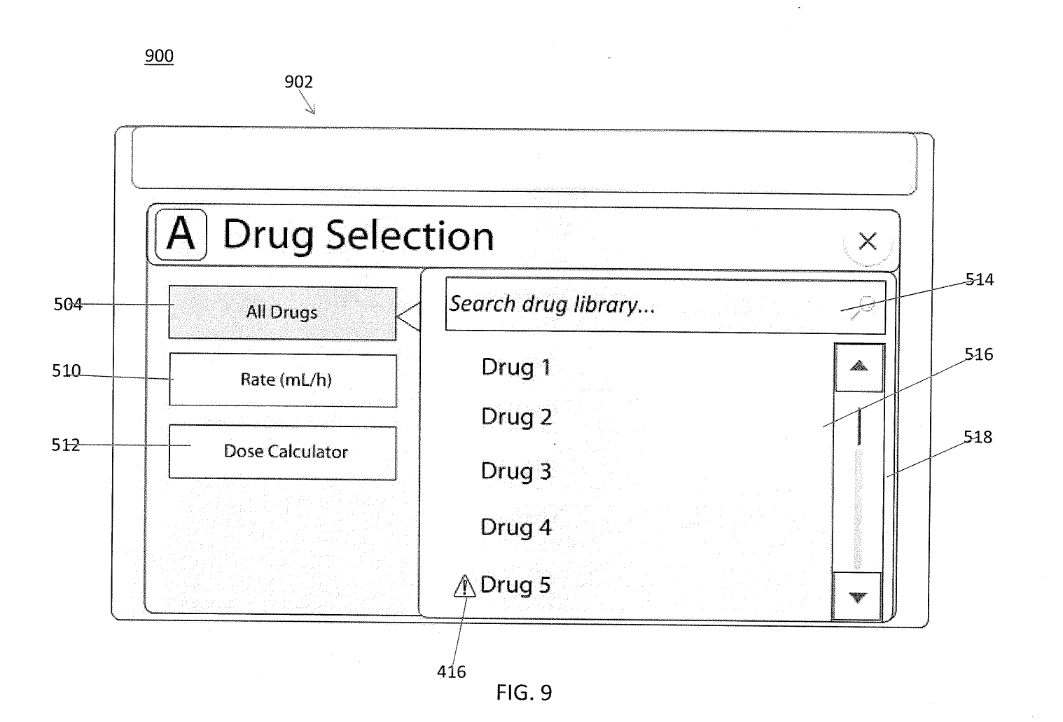

FIG. 9 is a diagram 900 illustrating a graphical user interface 902 displaying all the available drugs in the drugs list area 516 when the clinician selects the all drugs button 504.

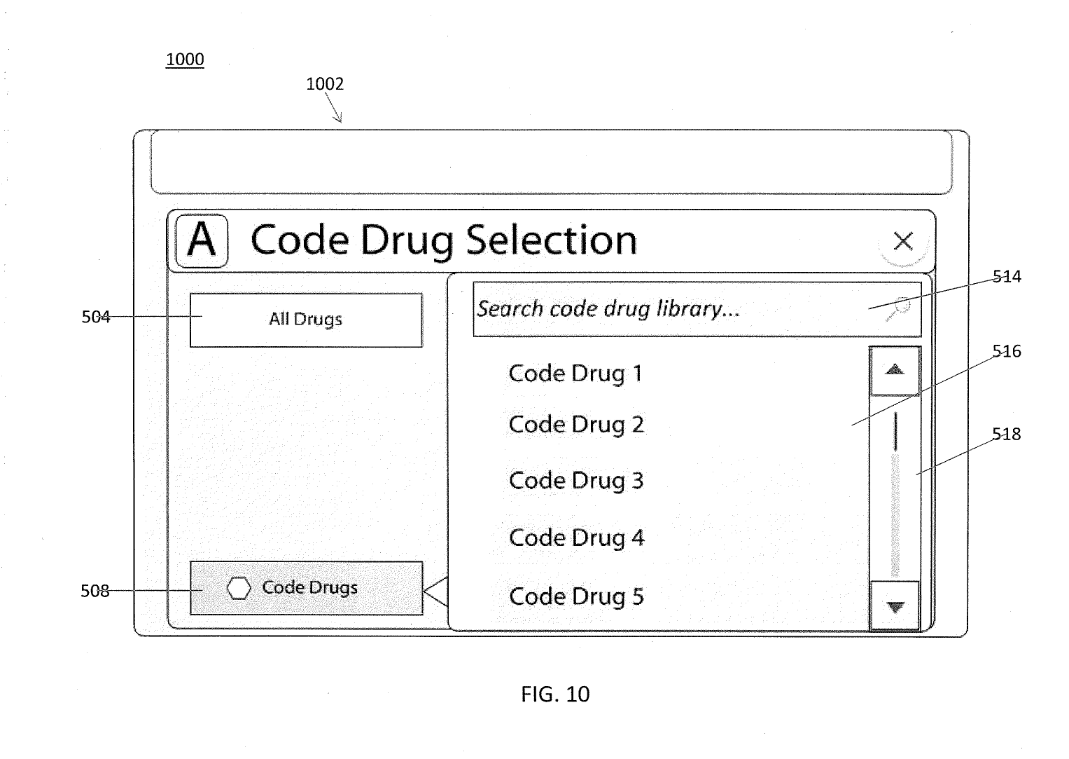

FIG. 10 is a diagram 1000 illustrating a graphical user interface 1002 displaying the code drugs in the drugs list area 516 when the clinician selects the code drugs button 508.

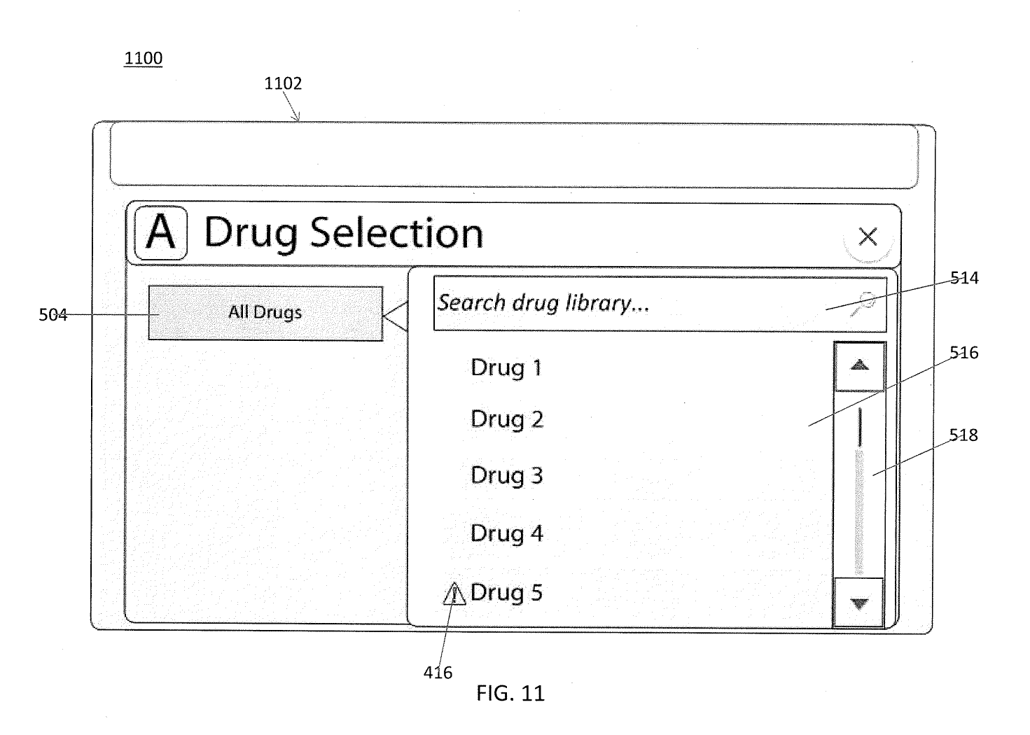

FIG. 11 is a diagram 1100 illustrating a graphical user interface 1102 displaying all drugs available in the hospital in the drugs list area 516 when the clinician selects the all drugs button 504.

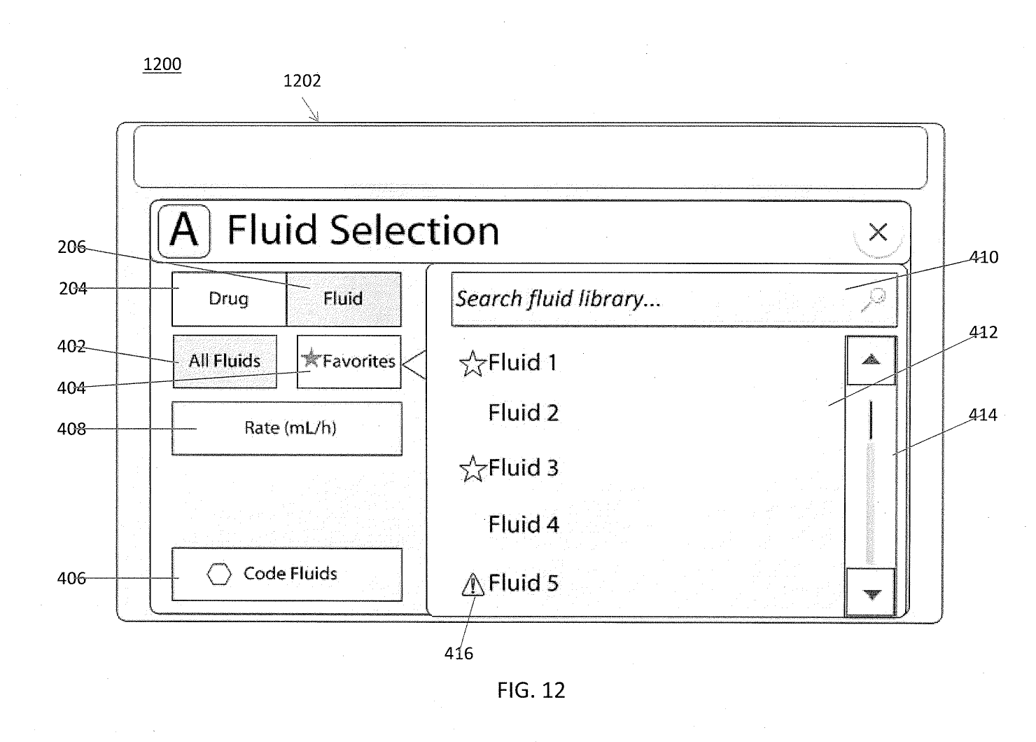

FIG. 12 is a diagram 1200 illustrating a graphical user interface 1202 displaying all fluids available in the hospital. The graphical user interface 1202 is an alternate example for the graphical user interface 402.

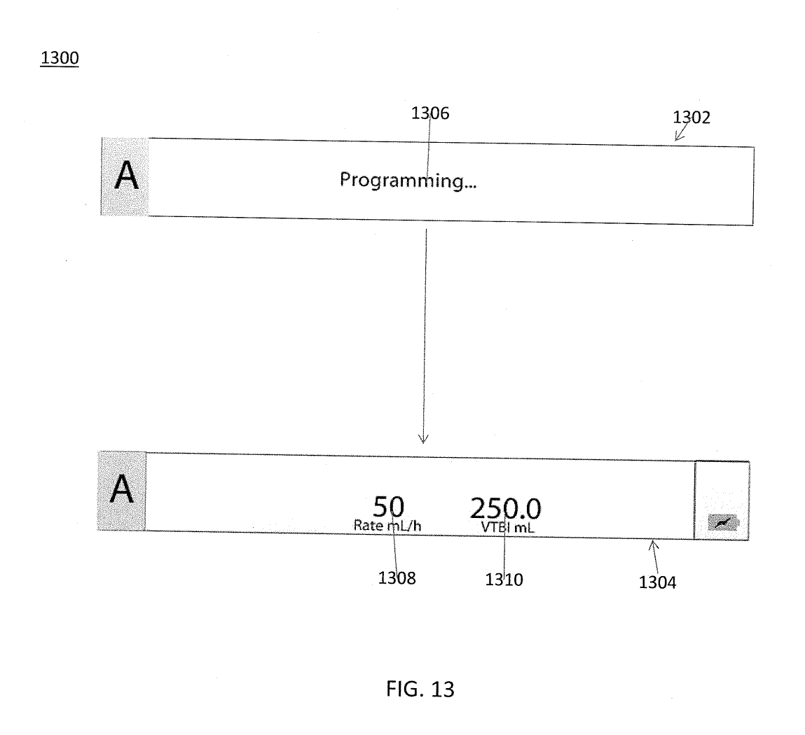

FIG. 13 is a diagram 1300 illustrating a transition from a first graphical user interface 1302 to a second graphical user interface 1304 on a user interface device 113 when the clinician specifies the rate of delivery and quantity of delivery. The user interface device 113 can include an infusion pump module display. Further, the user interface device 113 can also display drug dose, drug name, and concentration. When the clinician begins using the infusion system 102, the user interface device 113 can display the graphical user interface 1302. The graphical user interface 1302 can display a message 1306 (for example, “Programming . . . ”) until the infusion system 102 begins the delivery. When the clinician selects a medication and specifies the rate of delivery and quantity of delivery, the user interface device 113 can display the graphical user interface 1304. The graphical user interface 1304 can display the value 1308 of the rate of delivery and the value 1310 of the quantity of delivery until the selected medication is delivered.

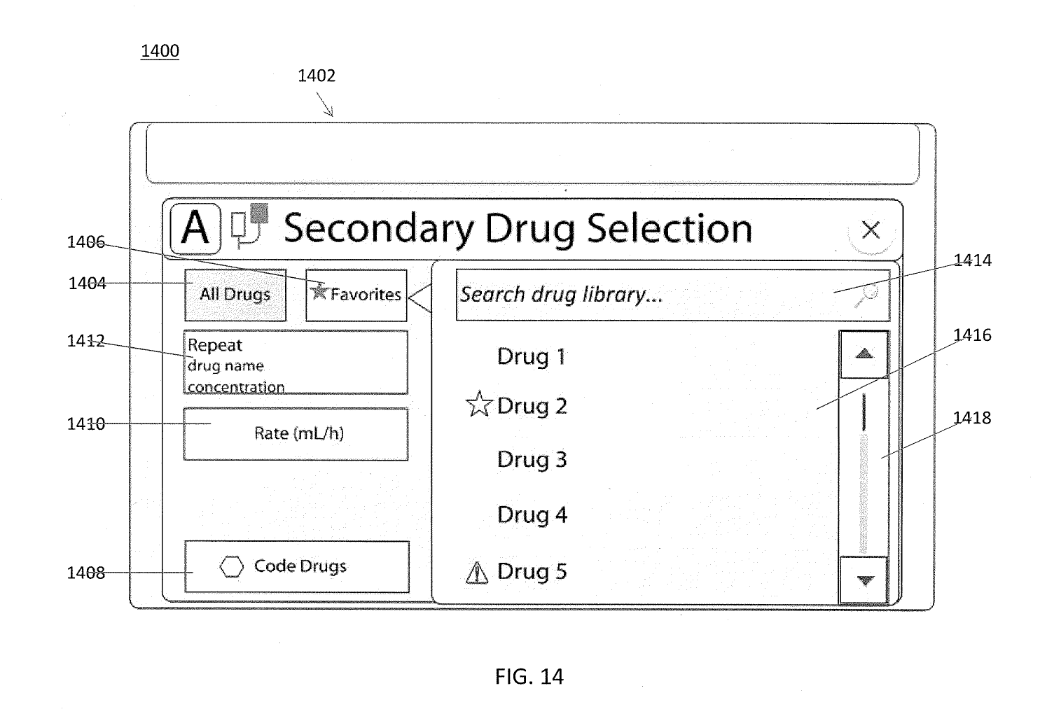

FIG. 14 is a diagram 1400 illustrating a graphical user interface 1402 allowing a clinician to select a dual rate sequential secondary drug for delivery from a list of all drugs. The user interface device 112 can provide an option to a clinician to select delivery of secondary drugs. If the clinician selects the option, the user interface device 112 can execute the graphical user interface 1402. The secondary drug could potentially be a drug that can be used only in emergency situations. The secondary drug can be delivered to a patient 104 prior to delivery of a primary fluid previously selected by the clinician on the user interface device 112.

The graphical user interface 1402 can display an all secondary drugs button 1404, a favorite secondary drugs button 1406, a code secondary drugs button 1408, a rate button 1410, a repeat button 1412, a search area 1414, a secondary drugs list area 1416, and an optional scrollbar 1418.

When the clinician clicks the all secondary drugs button 1406, the user interface device 112 can display all the secondary drugs in the secondary drugs list area 1416.

When the clinician clicks the favorite secondary drugs button 1406, the user interface device 112 can display only the favorite secondary drugs in the secondary drugs list area 1416. The favorite secondary drugs can characterize the secondary drugs that the clinician may prefer and may often provide to patients. The favorite secondary drugs can be displayed along with a common icon (for example, an icon showing a star) indicating that the associated secondary drug is a favorite secondary drug.

When the clinician clicks the code secondary drugs button 1408, the user interface device 112 can display only the code secondary drugs in the secondary drugs list area 1416. The code secondary drugs can characterize secondary drugs that can be usually delivered to patients diagnosed with a health condition treated in the healthcare area where the clinician works. The code secondary drugs can be displayed along with a common icon (for example, an icon showing a hexagon) indicating that the associated secondary drug is a code secondary drug.

The search area 1414 can allow the clinician to search for a secondary drug from the secondary drugs displayed in the secondary drugs list area 1416. In another implementation, the search area 1414 can allow the clinician to search for a secondary drug from a secondary drug library, which can include all the secondary drugs available in the hospital. The secondary drug library can be a portion of the medication library.

When the secondary drugs are displayed in the drugs list area 1416, the user interface device 112 can display secondary drugs in an alphabetical order. In another implementation, the user interface device 112 can display secondary drugs in the following order: favorite secondary drugs in an alphabetical order, code secondary drugs in an alphabetical order, and then remaining secondary drugs in alphabetical order. In yet another implementation, the clinician can specify any order for the secondary drugs displayed in the secondary drugs list area 1416.

When all the secondary drugs in the secondary drugs list area 1416 extend beyond a display area (for example, one page) that can be seen by the clinician, the graphical user interface 1402 can include a scrollbar that can be used to view all the secondary drugs in the secondary drugs list area 1416. In alternate implementations, the graphical user interface 1402 can allow the clinician to swipe a touchscreen to scroll though the list, as performed on some smart phones, such as an IPHONE.

When the clinician selects at least one secondary drug in the secondary drugs list area 1416, the user interface device 112 can allow the clinician to specify rates of delivery of the at least one selected secondary drug. The infusion system 102 can deliver the at least one secondary drug to the patient 104 at the specified rates.

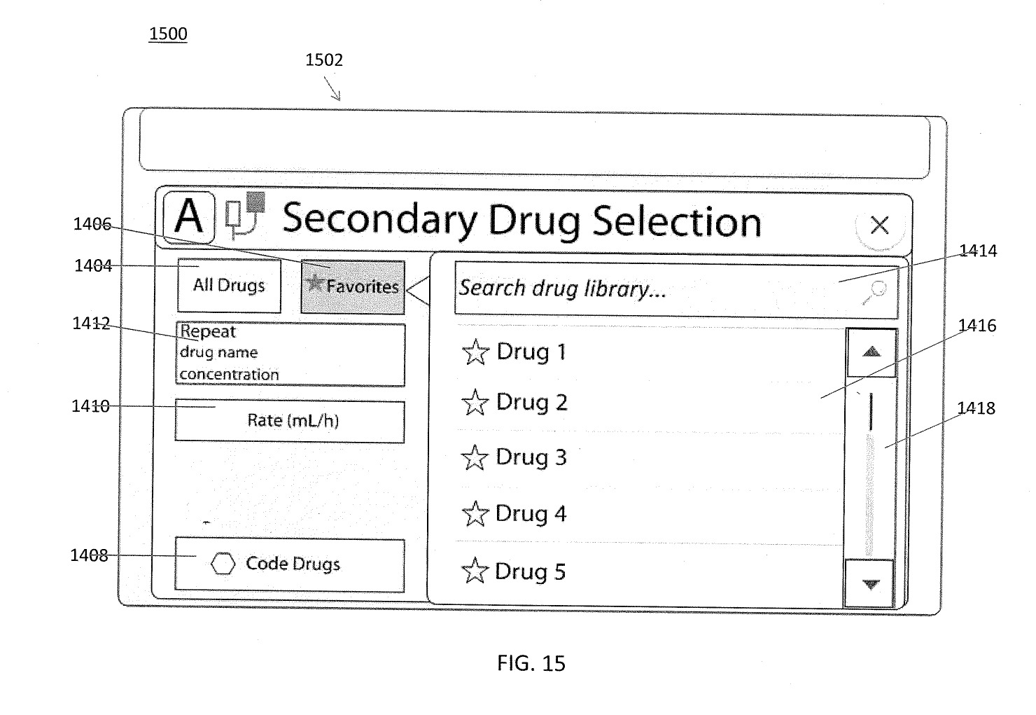

When the clinician selects the repeat button 1412, the clinician can specify whether the clinician desires to repeat a previous delivery of the selected secondary drug. FIG. 15 is a diagram 1500 illustrating a graphical user interface 1502 displaying favorite secondary drugs in the secondary drugs list area 1316 when the clinician selects the favorite secondary drugs button 1306. The user interface device 112 can execute the graphical user interface 1502.

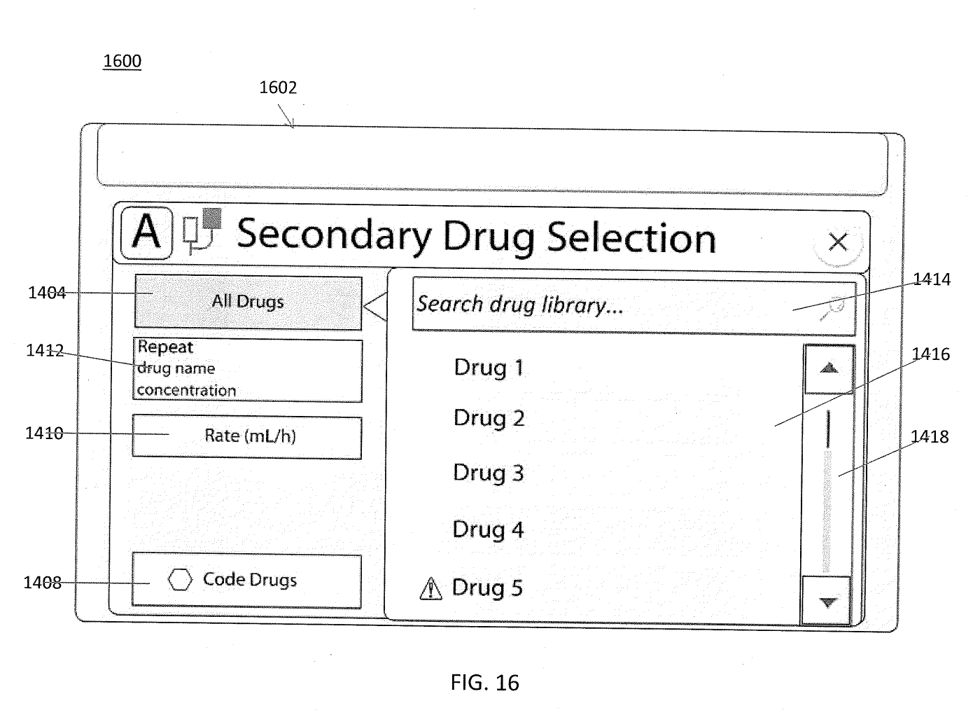

FIG. 16 is a diagram 1600 illustrating a graphical user interface 1602 displaying all secondary drugs in the secondary drugs list area 1316 when the clinician selects the all secondary drugs button 1304.

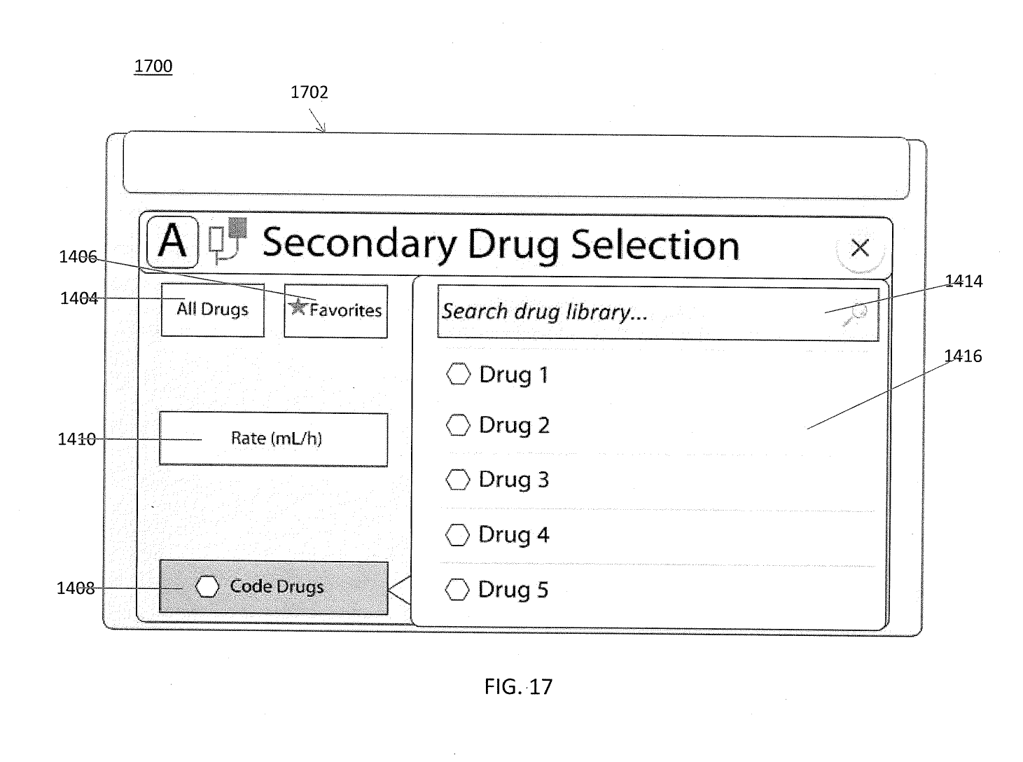

FIG. 17 is a diagram 1700 illustrating a graphical user interface 1702 displaying code secondary drugs in the secondary drugs list area 1316 when the clinician selects the code secondary drugs button 1316.

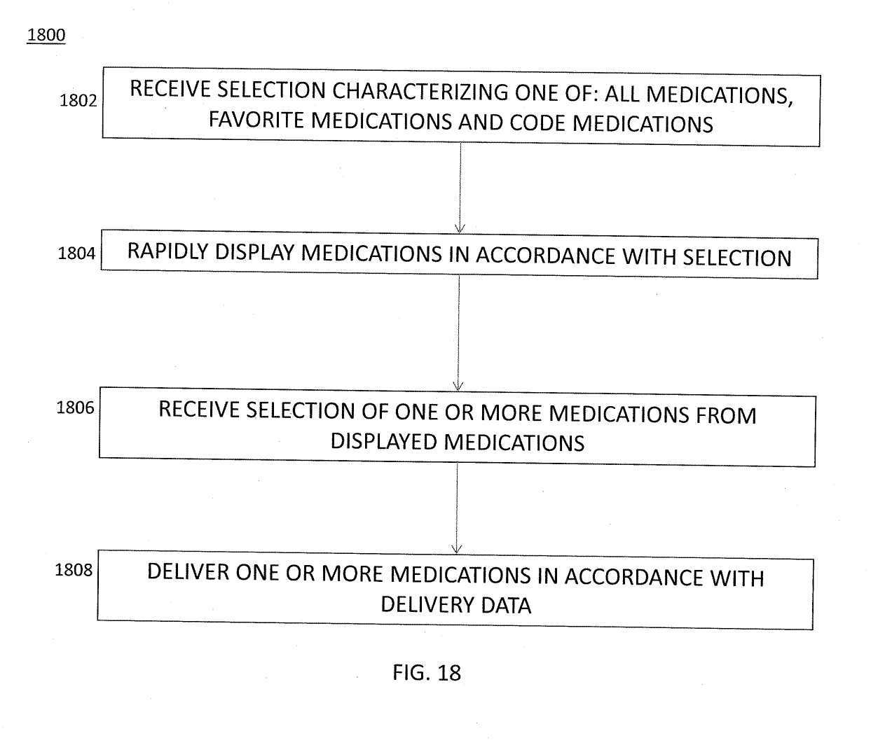

FIG. 18 is a flow-diagram 1800 illustrating a rapid display and subsequent delivery of medications required for treating the patient 104. The user interface device 112 can receive, at 1802 and from a clinician, a selection characterizing one of all medications, favorite medications, and code medications. The medications can be drugs (for example, pills) or fluid medications.

The user interface device 112 can retrieve, via a communication network, medications from a database based on the selection. The user interface device 112 displays, at 1804, the rapidly retrieved medications.

The database can include at least one of: (a) a mapping between each medication of a plurality of medications and corresponding healthcare area (for example, cardiac care unit, surgical ICU, where the patient is being treated, (b) a list of favorite medications and a list of favorite secondary medications for each healthcare area, and (c) a list of code medications for each healthcare area within each hospital. The storage of mappings and lists in the database can enable an advantageous rapid retrieval and subsequent display of medications at 1804. This storage of mappings and lists in the database also advantageously allows a display of only relevant medications, thereby saving time of the clinician. The communication network connecting the user interface device 112 and the database can include one or more of: a local area network, a wide area network, internet, intranet, Bluetooth network, infrared network, a wired network or other networks. In one implementation, the database can be external to the infusion system 102. In another implementation, the database can be embedded within the infusion system 102.

The user interface device 112 can receive, at 1806 and from the clinician, a section of one or more medications from the displayed medications. The user interface device can receive specifications of delivery data from the clinician. The delivery data can include one or more of: at least one infusion channel identifier identifying at least one infusion channel for the delivery of the selected medication by the delivery device, at least one of an initiation time and an initiation event that triggers to initiate the delivery of the selected medication, a quantity of the selected medication that is to be delivered, and a flow rate of the delivery of the selected medication. The infusion system 102 can deliver, at 1808, the selected one or more medications into the body of the patient 104 in accordance with the delivery data specified by the clinician. In other implementations, the clinician can manually deliver, at 1808, the medications to the patient 104 in accordance with the delivery data.

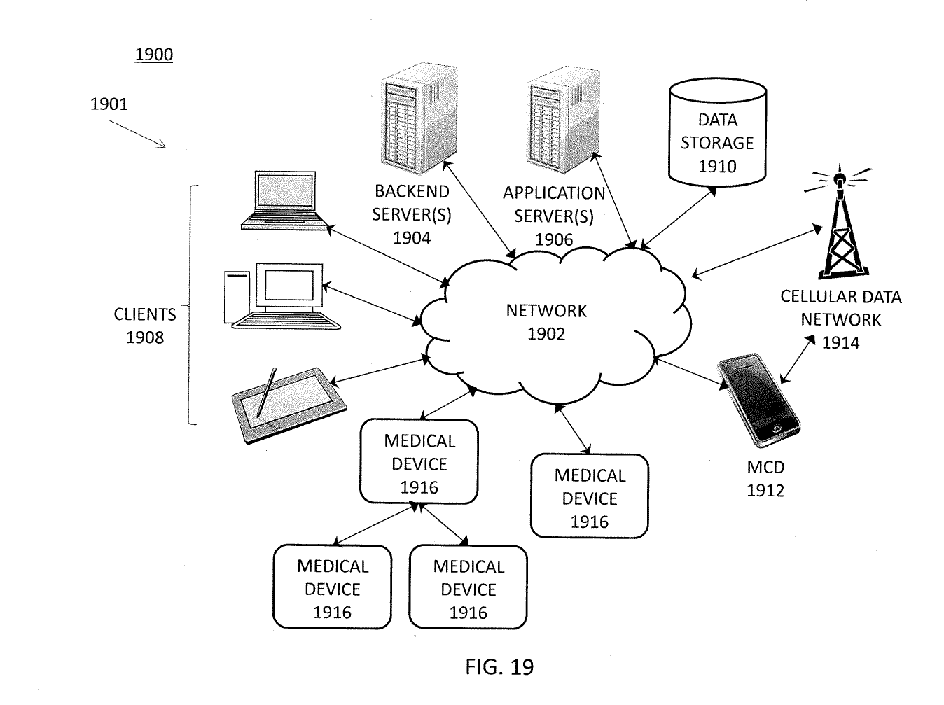

FIG. 19 is a system diagram 1900 illustrating a possible computing landscape 1901 for rapidly displaying and subsequently delivering medications required for treating the patient. The computing landscape 1901 can include the infusion system 102 and the medication storage and delivery system 122 within a healthcare environment, such as a hospital, a clinic, a laboratory, or any other environment. Various devices and systems, both local to the healthcare environment and remote from the healthcare environment, can interact via at least one computing network 1902. This computing network 1902 can provide any form or medium of digital communication connectivity (that is, wired or wireless) amongst the various devices and systems. Examples of communication networks include a local area network (“LAN”), a wide area network (“WAN”), and the Internet. In some cases, one or more of the various devices and systems can interact directly via peer-to-peer coupling (either via a hardwired connection or via a wireless protocol such as Bluetooth or WiFi). In addition, in some variations, one or more of the devices and systems communicate via a cellular data network.

In particular, aspects of the computing landscape 1901 can be implemented in a computing system that includes a back-end component (for example, as a data server 1904), or that includes a middleware component (for example, an application server 1906), or that includes a front-end component (for example, a client computer 1908 having a graphical user interface or a Web browser through which a user can interact with an implementation of the subject matter described herein), or any combination of such back-end, middleware, or front-end components. A client 1908 and servers 1904 and 1906 are generally remote from each other and typically interact through the communications network 1902. The relationship of the clients 1908 and servers 1904, 1906 arises by virtue of computer programs running on the respective computers and having a client-server relationship to each other. Clients 1908 can be any of a variety of computing platforms that include local applications for providing various functionalities within the healthcare environment. Example clients 1908 include, but are not limited to, desktop computers, laptop computers, tablets, and other computers with touch-screen interfaces. The local applications can be self-contained in that they do not require network connectivity and/or they can interact with one or more of the servers 1904, 1906 (for example, a web browser).

A variety of applications can be executed on the various devices and systems within the computing landscape such as electronic health record applications, medical device monitoring, operation, and maintenance applications, scheduling applications, billing applications and the like.

The network 1902 can be coupled to one or more data storage systems 1910. The data storage systems 1910 can include databases providing physical data storage within the healthcare environment or within a dedicated facility. In addition, or in the alternative, the data storage systems 1910 can include cloud-based systems providing remote storage of data in, for example, a multi-tenant computing environment. The data storage systems 1910 can also comprise non-transitory computer readable media.

Mobile communications devices 1912 can also form part of the computing landscape 1901. The mobile communication devices 1912 can communicate directly via the network 1902 and/or they can communicate with the network 1902 via an intermediate network such as a cellular data network 1914. Various types of communication protocols can be used by the mobile communication devices 1912 including, for example, messaging protocols such as SMS and MMS.

Various types of medical devices 1916 can be used as part of the computing landscape 1901. The medical devices 1916 can include one or more of the infusion system 102, and components within the infusion system 102. These medical devices 1916 can include, unless otherwise specified, any type of device or system with a communications interface that characterizes one or more physiological measurements of a patient and/or that characterize treatment of a patient. In some cases, the medical devices 1916 communicate via peer to peer wired or wireless communications with another medical device 1916 (as opposed to communicating with the network 1902). For example, the medical device 1916 can comprise a bedside vital signs monitor that is connected to other medical devices 1916, namely a wireless pulse oximeter and to a wired blood pressure monitor. One or more operational parameters of the medical devices 1916 can be locally controlled by a clinician, controlled via a clinician via the network 1902, and/or they can be controlled by one or more of a server 1904 and/or 1906, a client 1908, a mobile communication device 1912, and/or another medical device 1916.

The computing landscape 1901 can provide various types of functionality as can be required within a healthcare environment such as a hospital. For example, a pharmacy can initiate a prescription via one of the client computers 1908. This prescription can be stored in the data storage 1910 and/or pushed out to other clients 1908, a mobile communication device 1912, and/or one or more of the medical devices 1916. In addition, the medical devices 1916 can provide data characterizing one or more physiological measurements of a patient and/or treatment of a patient (for example, medical device 1916 can be an infusion management system, etc.). The data generated by the medical devices 1916 can be communicated to other medical devices 1916, the servers 1904 and 1906, the clients 1908, the mobile communication devices 1912, and/or stored in the data storage systems 1910.

Various implementations of the subject matter described herein can be realized/implemented in digital electronic circuitry, integrated circuitry, specially designed application specific integrated circuits (ASICs), computer hardware, firmware, software, and/or combinations thereof. These various implementations can be implemented in one or more computer programs. These computer programs can be executable and/or interpreted on a programmable system. The programmable system can include at least one programmable processor, which can be have a special purpose or a general purpose. The at least one programmable processor can be coupled to a storage system, at least one input device, and at least one output device. The at least one programmable processor can receive data and instructions from, and can transmit data and instructions to, the storage system, the at least one input device, and the at least one output device.

These computer programs (also known as programs, software, software applications or code) can include machine instructions for a programmable processor, and can be implemented in a high-level procedural and/or object-oriented programming language, and/or in assembly/machine language. As can be used herein, the term “machine-readable medium” can refer to any computer program product, apparatus and/or device (for example, magnetic discs, optical disks, memory, programmable logic devices (PLDs)) used to provide machine instructions and/or data to a programmable processor, including a machine-readable medium that can receive machine instructions as a machine-readable signal. The term “machine-readable signal” can refer to any signal used to provide machine instructions and/or data to a programmable processor.

To provide for interaction with a user, the subject matter described herein can be implemented on a computer that can display data to one or more users on a display device, such as a cathode ray tube (CRT) device, a liquid crystal display (LCD) monitor, a light emitting diode (LED) monitor, or any other display device. The computer can receive data from the one or more users via a keyboard, a mouse, a trackball, a joystick, or any other input device. To provide for interaction with the user, other devices can also be provided, such as devices operating based on user feedback, which can include sensory feedback, such as visual feedback, auditory feedback, tactile feedback, and any other feedback. The input from the user can be received in any form, such as acoustic input, speech input, tactile input, or any other input.

The subject matter described herein can be implemented in a computing system that can include at least one of a back-end component, a middleware component, a front-end component, and one or more combinations thereof. The back-end component can be a data server. The middleware component can be an application server. The front-end component can be a client computer having a graphical user interface or a web browser, through which a user can interact with an implementation of the subject matter described herein. The components of the system can be interconnected by any form or medium of digital data communication, such as a communication network. Examples of communication networks can include a local area network, a wide area network, internet, intranet, Bluetooth network, infrared network, or other networks.

The computing system can include clients and servers. A client and server can be generally remote from each other and can interact through a communication network. The relationship of client and server can arise by virtue of computer programs running on the respective computers and having a client-server relationship with each other.

Although a few variations have been described in detail above, other modifications can be possible. For example, the logic flows depicted in the accompanying figures and described herein do not require the particular order shown, or sequential order, to achieve desirable results. Other embodiments may be within the scope of the following claims.

What is claimed is:

1. A method comprising:
concurrently displaying, in a user interface device of an infusion system, a plurality of medications from a first medication set and a graphical user interface element, the graphical user interface element being associated with a plurality of medications from a second medication set, the second medication set being used for a clinical intervention for at least one patient-related code event;
receiving, at the user interface device, a clinician-generated input selecting the graphical user interface element; and
displaying, by the user interface device in response to the clinician-generated input, information characterizing one or more of the medications from the second medication set.

2. The method of claim 1, wherein the first medication set comprises medications specific for a healthcare area within a hospital.

3. The method of claim 1, wherein the graphical user interface element is a code medication button.

4. The method of claim 1, wherein the second medication set comprises code medications used to treat patients diagnosed with the at least one patient-related code event.

5. The method of claim 1, wherein the at least one patient-related code event comprises one of: a cardiac arrest, a respiratory arrest, a seizure, and a stroke.

6. The method of claim 1, wherein:
the at least one patient-related code event is cardiac arrest; and
the second medication set comprises Advanced Cardiovascular Life Support (ACLS) drugs including one or more of: epinephrine, vasopressin, amiodarone, procainamide, dopamine, sotalol, lidocaine, magnesium sulfate, adenosine, atropine, nitroglycerin, morphine, sodium bicarbonate, calcium blockers, angiotensin-converting-enzyme (ACE) inhibitors, glycoprotein inhibitors, anticonvulsants and fibrinolytic therapy medications.

7. The method of claim 1, wherein the displayed medications of the second medication set are retrieved for display from a database connected to the user interface device via a communication network.

8. The method of claim 7, wherein the database stores a mapping between the at least one patient-related code event and the second medication set.

9. The method of claim 1, further comprising:
receiving, at the user interface device, a selection by a clinician of at least one medication from the second medication set and a specification of delivery data for delivery of the at least one selected medication, the delivery data comprising a quantity of the at least one selected medication that is to be delivered, and a flow rate of the delivery of the at least one selected medication;
actuating, by a controller connected to the user interface device, a delivery of the at least one selected medication to a patient in accordance with the delivery data, the one or medications being delivered to the patient by at least one delivery device connected to the controller.

10. The method of claim 9, wherein:
the at least one delivery device obtains the selected medication from a medication storage device connected to the controller to deliver the medication to the patient;
the user interface device sends an identification of the selected one or more medications to a medication storage and delivery system that sends the selected at least one medication to the medication storage device; and
the user interface device is connected to the medication storage and delivery system via a communication network.

11. A system comprising:
a user interface device to: receive a preference for display of medications based on one of a plurality of medications, favorite medications from the plurality of medications, and code medications from the plurality of medications; display medications associated with the preference; receive a selection of a medication from the displayed medications and a delivery data;
a controller to receive the delivery data from the user interface device; and
a delivery device that is actuated by the controller to deliver the selected medication in accordance with the delivery data.

12. The system of claim 11, wherein:
the plurality of medications characterize all medications available in a hospital where the patient is being treated;
the favorite medications characterize medications that a clinician prefers and often provides to associated patients; and
the code medications characterize medications that are usually delivered to patients diagnosed with a health condition treated in a corresponding healthcare area where the clinician works and where the patient is being treated, the healthcare area being one of a plurality of healthcare areas within the hospital.

13. The system of claim 11, further comprising:
a database connected to the user interface device via a communication network, the database storing at least one of a mapping between each medication of the plurality of medications and a corresponding healthcare area of a plurality of healthcare areas where the patient is being treated, the favorite medications of a clinician using the user interface device, and the code medications for the corresponding healthcare area,
wherein the user interface retrieves medications associated with the preference of display from the database, the retrieved medications being displayed on a graphical user interface of the user interface device.

14. The system of claim 11, wherein the delivery data comprises at least one of: a quantity of the selected medication that is to be delivered and a flow rate of the delivery of the selected medication.

15. The system of claim 11, further comprising:
a medication storage device to store the selected medication, the delivery device obtaining the selected medication from the medication storage device to deliver the medication to the patient.

16. The system of claim 15, further comprising:
computational medication cabinets to receive the selection of the medication from the user interface device and deliver the selected medication to the medication storage device.

17. The system of claim 16, wherein the computational medication cabinets comprise a medication ordering system of a pharmacy.

18. An infusion pump comprising:
a user interface device to: receive a preference for display of medications based on one of a plurality of medications, favorite medications from the plurality of medications, and code medications from the plurality of medications; display medications associated with the preference; receive a selection of a medication from the displayed medications and a delivery data; and
a controller to receive the delivery data from the user interface device, the controller actuating a delivery device to deliver the selected medication in accordance with the delivery data.

19. The infusion pump of claim 18, wherein:
the user interface device is connected to a database via a communication network, the database storing at least one of a mapping between each medication of the plurality of medications and a corresponding healthcare area of a plurality of healthcare areas where the patient is being treated, the favorite medications of a clinician using the user interface device, and the code medications for the corresponding healthcare area,
wherein the user interface retrieves medications associated with the preference of display from the database, the retrieved medications being displayed on a graphical user interface of the user interface device.

20. The infusion pump of claim 18, wherein the delivery data comprises a quantity of the selected medication that is to be delivered and a flow rate of the delivery of the selected medication.

21. The infusion pump of claim 18, further comprising:
a medication storage device to store the selected medication, the delivery device obtaining the selected medication from the medication storage device to deliver the medication to the patient.

22. The infusion pump of claim 21, wherein the medication storage device receives the selected medication from a medication storage and delivery system.

23. The infusion pump of claim 22, wherein the medication storage and delivery system comprises computational medication cabinets that form a medication ordering system of a pharmacy.
